(19) United States
(12) Patent Application Publication
(10) Pub. No.: US 2012/0083592 A1
(43) Pub. Date: Apr. 5, 2012

(54) CONTINUOUS PREPARATION METHOD OF GINSENG GINSENOSIDES AND POLYSACCHARIDES

(76) Inventors: Zer-Ran Yu, Chiayi City (TW); Be-Jen Wang, Chiayi City (TW)

(21) Appl. No.: 12/894,387

(22) Filed: Sep. 30, 2010

(51) Int. Cl. C07H 1/08

(52) U.S. Cl. 536/6

(57) ABSTRACT

The present invention provides a continuous preparation method of ginseng ginsenosides and polysaccharides, whereby the ginseng extract liquor and supercritical solvent are poured continuously into a separation tank at 10-30 MPa and 40-60° C. as well as a preset flow rate; so the ginseng extract liquor can be separated in the separation tank to obtain ginsenosides and polysaccharides at different positions of the separation tank.

### BACKGROUND OF INVENTION

1. Field of the Invention

The present invention relates generally to a ginseng extraction technology, and more particularly to an innovative one which is involved with continuous preparation of ginseng ginsenosides and polysaccharides.

2. Description of Related Art

The scientific name of ginseng is Panax ginseng C. A. Meyer, a kind of Araliaceae perennial herb whose root is resembled like the human being. In many Asian countries, e.g.: China, South Korea and Japan, ginseng is widely used in traditional medical treatment, as recorded in Compendium of Materia Medica: “ginseng supplements the main organs, stabilizes the spirit and soul, eradicates the evil, improve the memory and extend the life span”; the health functions include: improving antidiuresis, lowering blood pressure, enhancing vascular and nerve center system, stimulating the immune system for stronger disease resistance, increasing metabolism and reducing the blood sugar; it is also commonly used in nourishing food against deficiency of qi, anemia, diabetes and neurasthenia. The main components in ginseng include: ginsenosides and polysaccharides, which can resist tumor activity and cytotoxicity, and inhibit the proliferation of tumor cells. Ginsenosides is considered as a main active component of ginseng. The research shows that, Rb1 in ginseng ginsenosides can lower the blood lipid, resist oxidation, inflammation and adjust the immunologic functions, whilst Rg1 in ginseng ginsenosides is believed to be capable of stimulating central nervous system, resisting fatigue, improving memory and learning functions as well as promoting angiogenesis. Thus, ginseng is one of most-commonly used and effective nutritious foods.

According to typical ginseng ginsenosides preparation technology, e.g.: ROC patent No. 200808971 “a method of producing ginseng ginsenosides Rg1 by free or immobilized enzyme”, carbohydrate is mainly used to inhibit β-glucosidase enzyme activity contained in free state enzyme; after adding ginseng ginsenosides Re, a reaction is made to form ginseng ginsenosides Rg1 in a complex way. According to polysaccharides extraction or separation technology, e.g. U.S. Pat. No. 6,555,527, methanol solvent and ethanol are firstly precipitated, then polysaccharides is separated using membrane dialysis and ion gel separation method, yet, the problems of solvent residues and toxicity are also encountered.

### CONTENT OF THE INVENTION

The primary objective of the present invention is to provide a continuous preparation method of ginseng ginsenosides and polysaccharides, whereby supercritical fluid technology is used to control the operating temperature and pressure, so as to separate continuously ginseng ginsenosides and polysaccharides from ginseng extract liquor, without the problems of solvent residues and toxicity.

The present invention provides a continuous preparation method of ginseng ginsenosides and polysaccharides; an operating pressure of 10-30 MPa and temperature of 40-60° C., ginseng extract liquor and supercritical solvent are poured at a preset flow rate into a separation tank, wherein ginseng extract liquor is separated to obtain ginsenosides and polysaccharides at different positions of the separation tank.

### BRIEF DESCRIPTION OF THE DRAWINGS

FIG. 1 is a sketch diagram of the continuous separation system of the preferred embodiment of the present invention; and

FIG. 2 is a diagram, showing the relationship of pressure, temperature and selectivity.

### DETAILED DESCRIPTION OF THE INVENTION

The following is a typical preferred embodiment of the present invention, which is described below in conjunction with the accompanying drawings:

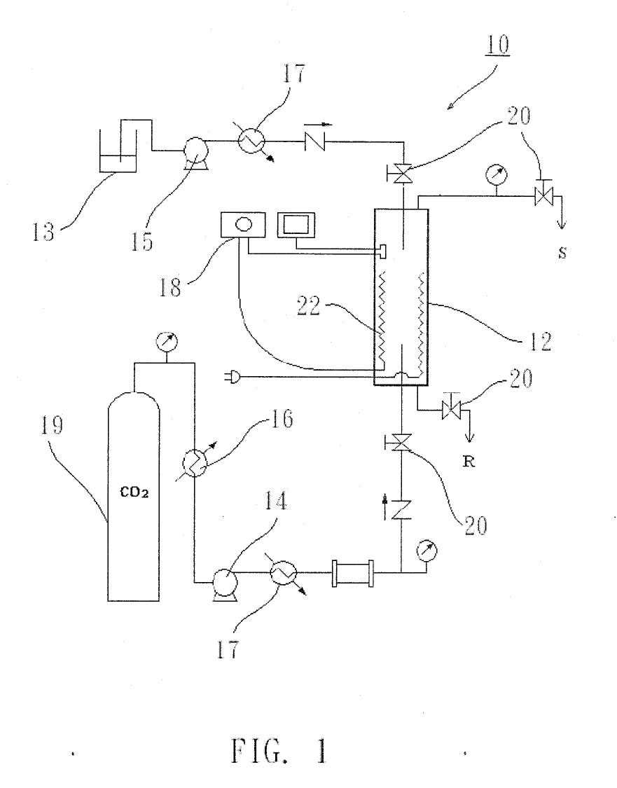

FIG. 1 depicts a preferred embodiment of a continuous preparation method of ginseng ginsenosides and polysaccharides of the present invention, wherein the supercritical fluid technology is used to prepare continuously ginseng active ingredient using a continuous separation system 10; the continuous separation system 10 comprises: a separation tank 12, a holding tank 13, a high-pressure metering pump 14, a reactant metering pump , a precooler 16, two preheaters 17, a temperature controller 18, a supercritical fluid vessel 19 and several valves 20; the separation tank 12 is composed of stainless steel tank body and monomer body, wherein an electric heater 22 is placed; the holding tank 13 is connected with reaction tank 12 to accommodate ginseng extract liquor; the high-pressure metering pump 14 is connected with supercritical fluid vessel 19; the reactant metering pump 15 is connected with holding tank 13; the precooler 16 is connected with supercritical fluid vessel 19; these two preheaters 17 are connected separately with high-pressure metering pump 14 and reactant metering pump 15; the temperature controller 18 is connected with electric heater 22, and the supercritical fluid vessel 19 is a CO2 steel cylinder.

According to the continuous preparation method of the present invention, ginseng extract liquor and supercritical solvent are poured at a preset flow rate into the separation tank 12, wherein ginseng extract liquor is separated to obtain ginsenosides and polysaccharides at different positions of the separation tank 12. In detail, the supercritical fluid vessel 19 is firstly opened, then high-pressure metering pump 14 and valves 20 are used to control the pressure of the separation tank 12, and the temperature controller 18 is used to control the temperature of the separation tank 12, with the operating conditions: 40-60° C., 10-30 MPa, flow rate of supercritical fluid 3-9 L/hr, flow rate of ginseng extract liquor 1-3 L/hr, as listed in Table 1; then the reactant metering pump 15 is started to add the ginseng extract liquor into the separation tank 12 in a controlled way; so the ginseng extract liquor can be separated in the separation tank 12 to form ginseng ginsenosides and polysaccharides. Ginseng ginsenosides is separated at top of separation tank 12 (S), and polysaccharides separated at bottom of separation tank 12 (R).

TABLE 1

| | | | | Ginseng extract |
|---|---|---|---|---|
| Ex. | Temp.(° C.) | Press.(MPa) | CO₂ (L/hr) | liquor(L/hr) |
| 1 | 60 | 20 | 9 | 1 |
| 2 | 60 | 30 | 3 | 2 |
| 3 | 50 | 10 | 9 | 2 |
| 4 | 50 | 30 | 6 | 1 |
| 5 | 50 | 20 | 3 | 3 |
| 6 | 40 | 30 | 9 | 3 |
| 7 | 40 | 10 | 3 | 1 |
| 8 | 60 | 10 | 6 | 3 |
| 9 | 40 | 20 | 6 | 2 |

After ginseng extract liquor is separated in the continuous separation system 10, ginseng ginsenosides at top of separation tank 12 and polysaccharides at bottom are collected, then the content is analyzed to calculate the separation efficiency of total ginsenosides (Ks), the separation efficiency of total polysaccharide (Kp) as well as selectivity. Separation efficiency of total ginsenosides (Ks) and separation efficiency of total polysaccharide (Kp) are defined below:

Ks=content of total ginsenosides at top of separation tank÷content of total ginsenosides at bottom of separation tank.

Kp=content of total polysaccharide at top of separation tank÷content of total polysaccharide at bottom of separation tank.

Selectivity:=Ks÷Kp.

When Ks=1, it indicates that the content of total ginsenosides at top of separation tank 12 is equal to the content of total ginsenosides at bottom of separation tank 12. When Kp=1, it indicates that the content of total polysaccharide at top of separation tank 12 is equal to the content of total polysaccharide at bottom of separation tank 12. Thus, if Ks>>1, it indicates that the content of total ginsenosides at separation tank 12 is easily separated at top of separation tank 12; if Kp<<1, it indicates that the content of total polysaccharide is easily separated at bottom of separation tank 12.

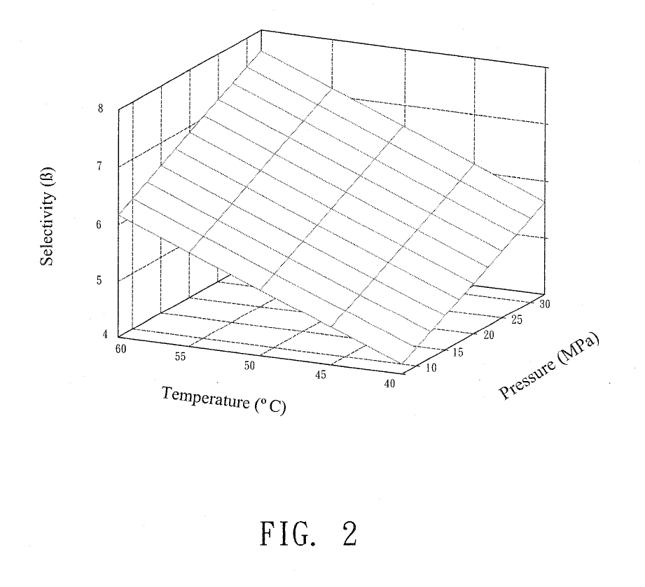

In addition, the separated value is subject to variable analysis by linear regression of SPSS statistical software. The statistical analysis result indicates that: CO2 solvent at flow rate (3, 6, 9 L/hr) and ginseng extract liquor at inlet rate (1, 2, 3 L/hr) do not affect the separation efficiency and selectivity (p>0.05), but the operating pressure and temperature will change the separation efficiency of total ginsenosides and total polysaccharides (p<0.05). Next, the separated value is analyzed using Response Surface Methodology of SPSS statistical software. The statistical analysis result indicates that: as shown in FIG. 2, when the pressure is higher (i.e. up to 30 MPa) and temperature is at 55-60° C., selectivity reaches 7.5, indicating that, if ginseng extract liquor containing 40 mg/g total ginsenosides and 30 mg/g total polysaccharide is separated in the separation tank 12, about 100 mg/g total ginsenosides is separated at top of the separation tank 12 (in FIG. 1, collector at position S), and about 160 mg/g total polysaccharide is separated at bottom of the separation tank 12 (in FIG. 1, collector at position R).

According to continuous preparation method of ginseng ginsenosides and polysaccharides of the present invention, ginseng extract liquor can be separated into active ingredient—ginseng ginsenosides and polysaccharides by using supercritical fluid technology and continuous separation system; as compared with conventional extraction and separation method, the present invention enables separation of ginsenosides and polysaccharides by only controlling the operating temperature and pressure, without the problems of solvent residue and toxicity; moreover, the separation efficiency and selectivity of ginsenosides and polysaccharides can be easily calculated, and the entire system can be operated continuously.

What is claimed is:

1. A continuous preparation method of ginseng ginsenosides and polysaccharides, ginseng extract liquor and supercritical solvent are poured at a preset flow rate into a separation tank, under operating conditions such as: 10-30 MPa and 40-60° C.; so, the ginseng extract liquor can be separated in the separation tank to obtain ginseng ginsenosides and polysaccharides at different positions of the separation tank.

2. The composition method defined in claim 1, wherein the ginseng extract liquor is poured at 1-3 L/hr into the separation tank, and the supercritical solvent is poured at 3-9 L/hr into the separation tank.

3. The composition method defined in claim 1, wherein the supercritical solvent is a CO2 fluid in supercritical state.

4. The composition method defined in claim 1, wherein the ginseng ginsenosides is separated at top of the separation tank, and the polysaccharides is separated at bottom of the separation tank.

5. The composition method defined in claim 1, wherein the optimum operating condition of the separation tank is: 30 MPa and 55-60° C.

6. The composition method defined in claim 1, wherein the continuous separation system comprises:
a separation tank, wherein an electric heater is placed;
a holding tank, connected with the separation tank to accommodate ginseng extract liquor;
a supercritical fluid vessel, connected with the separation tank to provide supercritical fluid;
a high-pressure metering pump, connected between the supercritical fluid vessel and separation tank;
a reactant metering pump, connected between the holding tank and separation tank;
a precooler, connected between the supercritical fluid vessel and high-pressure metering pump;
two preheaters, connected separately between the high-pressure metering pump, reactant metering pump and separation tank;
a temperature controller, connected with the electric heater; and
several valves, arranged between two preheaters and the separation tank.

7. The system defined in claim 6, wherein said separation tank is composed of a tank body and a monomer body in the tank body; the electric heater is set in the tank body.

8. The system defined in claim 7, wherein said separation tank is made of stainless steel.

9. The system defined in claim 6, wherein the supercritical fluid vessel is used to accommodate supercritical CO2 fluid.
